(19) United States
(12) Patent Application Publication
(10) Pub. No.: US 2020/0370120 A1
(43) Pub. Date: Nov. 26, 2020

(54) REFERENCE MATERIAL FOR NON-INVASIVE PRENATAL TESTING AND MANUFACTURING METHOD THEREOF

(71) Applicant: Beijing Hospital, Beijing (CN)

(72) Inventors: Jinming Li, Beijing (CN); Jiansheng Ding, Beijing (CN); Rui Zhang, Beijing (CN)

(21) Appl. No.: 16/959,431

(22) PCT Filed: Nov. 27, 2018
(86) PCT No.: PCT/CN2018/117761

§ 371 Date: Jun. 30, 2020

(30) Foreign Application Priority Data
Nov. 12, 2018 (CN) 201811338459.7

(51) Int. Cl. C12Q 1/6883; C12Q 1/6806

(52) U.S. Cl. CPC C12Q 1/6883; C12Q 2600/166; C12Q 1/6806

(57) ABSTRACT

The invention relates to a method for preparing a simulated maternal cfDNA component for non-invasive prenatal testing, using a DNA fragmentation factor to digest a nucleus for simulating a maternal sample cell line, so as to obtain a simulated maternal cfDNA (short nucleic acid fragment) component. The reference material for non-invasive prenatal testing prepared by mixing different chromosome aneuploid cfDNA fragments with the normal chromosome number cfDNA fragments obtained by the present invention provides a method for manufacturing a reference material for internal quality control and external quality assessment. This method of making a reference material by mixing chromosome aneuploid cfDNA fragments and normal chromosome number cfDNA fragments produced with different enzyme digestions is the first case in the art. This method can also be used to make a series of mixed cfDNA sample reference materials for other noninvasive testing.

### FIELD OF THE INVENTION

The invention discloses a reference material for non-invasive prenatal testing and a preparation method thereof, and belongs to the fields of clinical laboratory science and biotechnology.

### BACKGROUND OF THE INVENTION

Fetal chromosome aneuploids are mainly caused by abnormalities of chromosome number or structure, and clinically, trisomy 21 (Down syndrome), trisomy 18 (Edwards syndrome), and trisomy 13 (Patau syndrome) are most common and most likely prone to occur. Children patients usually show mental retardation, stunted growth, or reproductive disorders in adulthood. The incidence of this type of diseases increases with the increase of age of the pregnant woman, and there currently is no effective treatment. Therefore, extensive prenatal screening and prenatal diagnosis of chromosomal diseases are of great significance for reducing birth defects, achieving eugenics and improving population quality.

Existing prenatal diagnostic techniques include invasive detection methods and non-invasive detection methods, the former such as amniocentesis or chorionic membrane sampling to obtain fetal tissue and perform karyotype analysis, but the disadvantage thereof is that they are all invasive and have risks such as miscarriage; while the traditional non-invasive testing methods widely used in home and abroad include screening of pregnant women's serum biochemical markers and ultrasonography, but these methods usually have low accuracy, and both of the false positive rate and false negative rate of their test results are high, in addition, a lot of false positive results bring great anxiety to pregnant women and their families, which may cause neurodevelopment and mental disorders in their offspring.

In 1997, Professor Lu Yuming discovered that cell-free fetal DNA (cffDNA) was contained in the plasma of pregnant women, which opened a new era in the field of non-invasive prenatal testing. At present, non-invasive prenatal testing (NIPT) based on fetal chromosome aneuploid of cell-free DNA has been widely used in clinical practice. At present, the most commonly used detection method is high-throughput sequencing (next-generation sequencing, NGS), which specifically comprises extracting cell-free DNA (cfDNA) in the plasma blood of pregnant women, analyzing a large amount of data generated by NGS, calculating a ratio of the reads for single chromosome sequencing to the total reads of all chromosomes, then comparing the ratio to the corresponding ratio of normal pregnant women's plasma in the reference database, and determining whether the fetus has aneuploid abnormality by a relevant statistical method. Compared to traditional detection methods, this method aims at the detection of trace cell-free DNA in maternal blood, and thus is safer, simple and convenient, and stable, and has significantly improved accuracy and sensitivity.

Although non-invasive prenatal testing of cell-free DNA has high accuracy and good application prospects, it still faces challenges in method development and laboratory testing. First of all, for non-invasive prenatal testing, it is necessary to be able to accurately analyze trace amount of residual cell-free fetal DNA in the presence of interference with maternal genetic information. In the detection of fetal genetic abnormalities, comparisons of small differences are performed in most of cases, which have high requirements for the accuracy and repeatability of detection. Second, the complexity of the non-invasive prenatal testing process and numerous steps may affect the results, including the extraction efficiency of cell-free DNA, factors affecting accuracy such as library quality, coverage and uniformity of sequencing, correction of GC bias, bioinformatics algorithm. Third, the current simulated maternal sample is prepared by a method of randomly breaking DNA by ultrasonic, while such simulated cfDNA sample has randomness, the sequence information contained in the fragment, the fragment length and distribution are not consistent with the real sample; and the most common simulated cfDNA sample obtained by the MNase digestion has a fragment length of about 146 bp, which is relatively shorter as compared to the length of maternal cfDNA component in real plasma sample (the main peak is distributed at 166 bp), so that MNase method can not prepare a sample simulating the maternal cfDNA components.

Therefore, the current non-invasive prenatal testing is in urgent need of standardization, and the reference materials consistent with real clinical samples are the most important preconditions for standardization, methodological verification, internal quality control and external quality assessment.

### SUMMARY OF THE INVENTION

The first technical problem to be solved by the present invention is to provide a method for preparing a simulated maternal cfDNA component for non-invasive prenatal testing.

The second technical problem to be solved by the present invention is to provide a simulated maternal cfDNA component for non-invasive prenatal testing.

The third technical problem to be solved by the present invention is to provide a method for preparing a reference material for non-invasive prenatal testing.

The fourth technical problem to be solved by the present invention is to provide a reference material that can be applied to non-invasive prenatal testing.

To achieve the above objective, the present invention adopts the following technical solutions:

A method for preparing a simulated maternal cfDNA component for non-invasive prenatal testing, comprising using a DNA fragmentation factor (DFF) to digest a nucleus for simulating a maternal sample cell line, so as to obtain a simulated maternal cfDNA (short nucleic acid fragment) component. The simulated cfDNA component can be used to simulate a maternal cfDNA component in a plasma of a pregnant woman in clinic.

DFF (DNA fragmentation factor) is a key nuclease involved in DNA fragmentation during apoptosis process. DFF itself does not have nuclease digestion activity, but it can generate two subunits in size of 40 kilodaltons and 45 kilodaltons under the action of apoptotic proteases (such as apoptotic protease-3), wherein the 40-kilodaltons subunit, i.e., DFF fragmentation factor 40 (DNA fragmentation factor 40 kDa, DFF40), has endogenous nuclease digestion activity, and can specifically cleave the ligation region between nucleosomes, thereby finally generating DNA fragments that are mainly of single nucleosome length.

By constructing a DFF prokaryotic expression plasmid, transforming a competent cell, a complete DFF is expressed and purified, the purified DFF is processed by apoptotic protease-3 (Caspase-3) under appropriate reaction conditions to produce DFF40, and the DFF40 can digest a corresponding chromatin substrate, so that the process of DNA fragmentation during the last phase of apoptosis is simulated in vitro. The above specific methods for the construction of recombinant DFF expression plasmid, the expression and purification of DFF are all prior art, see U.S. Pat. No. 6,165,737, DNA fragmentation factor involved in apoptosis (U.S. Pat. No. 6,165,737, DNA FRAGMENTATION FACTOR INVOLVED IN APOPTOSIS).

The method for preparing a simulated maternal cfDNA component for non-invasive prenatal testing is characterized in that the simulated maternal sample cell line is a human cell line with a normal number of chromosomes.

The human cell line is an immortalized cell line, for example, a human B lymphocyte.

The method for digesting a nucleus of a maternal sample cell line comprises:

(1) taking about 1×10⁷ nuclei of the simulated maternal sample cell line, washing the nuclei with 1 ml of buffer A, repeating the washing twice, centrifuging at 200 g at 4° C. for 5 min, discarding a supernatant, and resuspending the nuclei with 100 μl of buffer A to obtain a cell nuclear solution;

in which the buffer A has components of: 20 mM Hepes-KOH (pH 7.5), 10 mM KCl, 1.5 mM MgCl₂, 1 mM sodium EDTA, 1 mM sodium EGTA, 1 mM DTT, 0.1 mM PMSF;

(2) preparing a digestion reaction system and incubating at 37° C. for 2 hours;

the digestion reaction system comprises: 30 μL of DFF solution at a concentration of 0.585 mg/ml, 45 μL of human caspase-3 at a concentration of 95 ng/μL, 28 μL of buffer A, and 10 μL of the nuclear solution obtained in step (1);

(3) adding 25 μl of a reaction stop solution to terminate the reaction, mixing by inversion, and incubating at 50° C. for 1 h; the composition of the stop solution is 0.6% SDS, 50 mM EDTA, and 6 mg/ml proteinase K;

(4) determining a DNA concentration in the reaction product and observing the size distribution of nucleic acid fragments by capillary electrophoresis.

In another aspect, the present invention provides a stimulated maternal cfDNA component for non-invasive prenatal testing.

The simulated maternal cfDNA component is prepared for non-invasive prenatal testing by the method described above. The length of the simulated maternal cfDNA is about 160 bp, which is close to the length of the maternal cfDNA in the clinical pregnant woman's plasma that is 160-170 bp. The preparation of the simulated maternal cfDNA component in the present patent provides a universal method for preparing a simulated normal healthy human plasma cfDNA sample. At the same time, the simulated maternal cfDNA component is not only used for preparing non-invasive prenatal fetal chromosome aneuploid detection reference material, but also used in other cfDNA-based non-invasive detection fields, such as preparing a cfDNA reference material for detecting a single-gene genetic disease.

In another aspect, the present invention provides a method for preparing a reference material for non-invasive prenatal testing, comprising digesting a nucleus of a simulated fetal sample cell line with a micrococcal nuclease (MNase) to obtain a simulated fetal cfDNA component (used for simulating fetal cfDNA component in the plasma of pregnant women in clinic); digesting a nucleus of a simulated maternal sample cell line with a DNA fragmentation factor (DFF) to obtain a simulated maternal cfDNA component; mixing the simulated fetal cfDNA component and the simulated maternal cfDNA component at a mass ratio of 1:99 to 30:70 to obtain a simulated mixed cfDNA component; adding the simulated mixed cfDNA component to an artificial plasma to make a reference material with a simulated mixed cfDNA concentration of 1 to 100 ng/ml.

The simulated maternal sample cell line is a human cell line with a normal number of chromosomes, and the simulated fetal sample cell line is a chromosome aneuploid-positive human cell line or a human cell line with a normal number of chromosomes.

The human cell line is an immortalized cell line.

The artificial plasma is a simulated body fluid containing a human plasma albumin at a concentration of 5%.

The method for digesting the nucleus of the simulated fetal sample cell line with micrococcus nuclease comprises the follow steps:

(1) taking 1×10⁷ nuclei of the simulated fetal sample cell line, washing the nuclei with 2 to 3 ml of MNase reaction buffer, repeatedly washing for 3 times, centrifuging at 120 g at 4° C. for 10 min, discarding the supernatant, and using 100 μl of MNase reaction buffer to resuspend the nuclei to obtain a nuclear suspension;

in which the MNase reaction buffer has components of: 1×Micrococcal Nuclease reaction buffer, 9% (v/v) 2-ME, 1 tablet/10 ml protease inhibitor, 100 μg/ml BSA;

(2) adding 300 U of MNase to the above nuclear suspension and incubating in a 37° C. water bath for 10 min;

(3) adding 20 μl of MNase stop solution to the reaction tube, standing at room temperature for 5 min; the MNase stop solution having components of 250 mmol/L EDTA, 250 mmol/L EGTA;

(4) determining the DNA concentration in the reaction product, and observing the size distribution of the nucleic acid fragments by capillary electrophoresis;

the method for digesting the nucleus of the simulated maternal sample cell line comprises the following steps:

(1) taking about 1×10⁷ nuclei of the simulated maternal sample cell line, washing the nuclei with 1 ml of buffer A, repeating the washing twice, centrifuging at 200 g at 4° C. for 5 min, discarding the supernatant, and resuspending the nuclei with 100 μl of buffer A to obtain a nuclear solution;

the buffer A has components of 20 mM Hepes-KOH (pH 7.5), 10 mM KCl, 1.5 mM MgCl₂, 1 mM sodium EDTA, 1 mM sodium EGTA, 1 mM DTT, 0.1 mM PMSF;

(2) preparing a digestion reaction system and incubating at 37° C. for 2 hours;

the digestion reaction system comprises: 30 μL of DFF solution at a concentration of 0.585 mg/ml, 45 μL of human caspase-3 at a concentration of 95 ng/μL, 28 μL of buffer A, and 10 μL of the nuclear solution obtained in step (1);

(3) adding 25 μl of reaction stop solution to terminate the reaction, mixing by inversion, and incubating at 50° C. for 1 h; the stop solution has composition of 0.6% SDS, 50 mM EDTA, 6 mg/ml proteinase K;

(4) determining the DNA concentration in the reaction product and observing the size distribution of the nucleic acid fragments by capillary electrophoresis.

In another aspect, the present invention provides a reference material for non-invasive prenatal testing.

A reference material prepared by any one of the methods described above for non-invasive prenatal testing, the simulated maternal sample cell line is one or more selected from normal chromosome number human cell line GM12878, normal chromosome number human cell line GM23087, and normal chromosome number human cell line AG09387; the simulated fetal sample cell line is one or more selected from 21-trisomy positive human cell line AG09394, 18-trisomy positive human cell line GM02732, 13-trisomy positive human cell line GM02948, and normal chromosome number human cell line GM23086.

The cell line in the present invention includes, but is not limited to, the above-mentioned cell lines. Among them, GM12878 is a standard cell line, that is, a cell line which DNA sequence has been clearly determined. GM23087, GM23086, and AG09387 are all cell lines which karyotypes have been determined. AG09394, GM02732, and GM02948 are all cell lines which karyotypes have been determined.

More specifically, the present invention provides a reference material for non-invasive prenatal testing, which is composed of 3 positive mixed samples and 1 normal control sample; each positive mixed sample and normal control sample are composed of a simulated maternal cfDNA component, a simulated fetal cfDNA component and an artificial plasma, wherein the mass percentage ratio of the simulated fetal cfDNA component to the simulated maternal cfDNA component is 1:99 to 30:70, the artificial plasma is a simulated body fluid containing human plasma albumin at a concentration of 5%, and the concentration of the sum of the simulated fetal cfDNA component and the simulated maternal cfDNA component in artificial plasma is 1 to 100 ng/ml;

The 3 positive mixed samples are 21-trisomy positive mixed sample, 18-trisomy positive mixed sample and 13-trisomy positive mixed sample, respectively; in the 21-trisomy positive mixed sample, the simulated maternal cfDNA component is cfDNA of normal chromosome number human cell line AG09387 obtained by DFF digestion, and the simulated fetal cfDNA component is cfDNA of 21-trisomy positive human cell line AG09394 obtained by MNase digestion; in the 18-trisomy positive mixed sample, the simulated maternal cfDNA component is cfDNA of normal chromosome number human cell line GM12878 obtained by DFF digestion, and the simulated fetal cfDNA component is cfDNA of 18-trisomy positive human cell line GM02732 obtained by MNase digestion; in the 13-trisomy positive mixed sample, the simulated maternal cfDNA component is cfDNA of normal chromosome number human cell line GM12878 obtained by DFF digestion, and the simulated fetal cfDNA is cfDNA of 13-trisomy positive human cell line GM02948 obtained by MNase digestion;

In the normal control sample, the simulated maternal cfDNA component is cfDNA of normal chromosome number human cell line GM23087 obtained by DFF digestion, and the fetal cfDNA component is cfDNA of normal chromosome number human cell line GM23086 obtained by MNase digestion.

In the present invention, normal chromosome number human cell line GM12878, or normal chromosome number human cell line GM23087, or normal chromosome number human cell line GM23086, or normal chromosome number human cell line AG09387, and 21-trisomy positive human cell line AG09394, or 18-trisomy positive human cell line GM02732, or 13-trisomy positive human cell line GM02948 are selected, the cells are cultured to a certain number and their nuclei are extracted. The nuclei derived from different chromosome aneuploid positive cell lines or normal chromosome number human cell line GM23086 are digested by MNase to produce corresponding cfDNA fragments; while the nuclei derived from normal chromosome number human cell line GM12878 or GM23087 or AG09387 are digested by DFF to produce corresponding cfDNA fragments, and the corresponding DNA fragments were recovered; each of the cfDNA fragments obtained by MNase digestion of the nuclei and the corresponding cfDNA fragments obtained by DFF digestion of the nucleus are mixed at a certain ratio, the human plasma albumin at a concentration of 5% is mixed with the simulated body fluid to prepare an artificial plasma (human plasma albumin concentration (w/v) in the artificial plasma has a unit of g/100 ml), then the above mixed cfDNA fragments and the simulated plasma are mixed at a certain concentration to obtain a mixed cfDNA plasma sample, and a sample tray of NIPT reference materials is established containing 3 chromosome aneuploid positive mixed samples and 1 normal control sample composed of cfDNA fragments derived from normal chromosome number human cell line.

The reference material cfDNA prepared by mixing the chromosome aneuploid cfDNA fragment produced by MNase digestion and the normal chromosome number cfDNA fragment produced by DFF digestion has high quality and good stability, and can artificially control and select the required different chromosome aneuploid types, the cfDNA fragments produced by different enzyme digestions are consistent with the various biochemical characteristics of maternal and fetal-derived cfDNAs in the plasma of real pregnant women. At the same time, mother-child pair samples can be prepared by selecting cell line sources, which overcomes the deficiency of traditional NIPT reference materials. The reference material produced by mixing the chromosome aneuploid cfDNA fragments with the normal chromosome number cfDNA is easy to operate, has a short synthesis cycle, and can be produced in large quantities. The reference material produced in this study can be used as a reference material for non-invasive prenatal testing, can be widely used in methodology validation, internal quality control and external quality assessment, and has good repeatability and consistency, which is beneficial to achieve standardization of non-invasive prenatal testing.

The innovation of the present invention lies in:

1. The present invention discovers and confirms for the first time that the nucleus of a simulated maternal sample cell is digested with DFF to obtain a reference material that stimulates the mother-derived cfDNA in a real clinical pregnant woman plasma sample, so that the problem in the prior art that a detectable cfDNA reference material as desired cannot be obtained by enzymatic digestion of other substances is completely solved, and the large-scale preparation of artificially simulated plasma cfDNA standardized testing reference material is realized.

2. The mixed cfDNA plasma sample reference material obtained by the present invention can simulate a real clinical specimen. MNase digestion of nuclei produces cfDNA fragments similar to fetal cfDNA in pregnant woman's plasma, and DFF digestion of nuclei produces cfDNA fragments similar to maternal cfDNA in pregnant woman's plasma, and the above two are mixed at a certain ratio to obtain mixed cfDNA fragments similar to pregnant women's plasma cfDNA. Therefore, we can mix the cfDNA fragments with the prepared artificial plasma to obtain a reference material that simulates the real pregnant woman's plasma, which can be used for a series of methodological verification, internal quality control and external quality assessment. The reference material prepared by mixing chromosome aneuploid cfDNA fragments and normal chromosome number cfDNA fragments in our research can be used as a reference material for non-invasive prenatal testing. This method of making a reference material by mixing chromosome aneuploid cfDNA fragments and normal chromosome number cfDNA fragments produced with different enzyme digestions is the first case in the art. This method can also be used to make a series of mixed cfDNA sample reference materials for other noninvasive testings.

The following describes the present invention with reference to the accompanying drawings and specific embodiments, so that the public can better understand the content and application of the present invention, and does not limit the present invention in any way. Any equivalent replacement made in accordance with the disclosure of the present invention belongs to the protection scope of the present invention.

### BRIEF DESCRIPTION OF THE DRAWINGS

FIG. 1 shows the electrophoresis result of DFF prepared in Example 2.

FIG. 2A shows the Agilent 2100 electrophoresis peak diagram of cfDNA fragments obtained by digesting cell nuclei with MNase (Micrococcal nuclease, MNase).

FIG. 2B shows the Agilent 2100 electrophoresis peak diagram of cfDNA fragments obtained by digesting cell nuclei with DFF (DNA fragmentation factor, DFF).

FIG. 2C shows the Agilent 2100 electrophoresis peak diagram of the mixed cfDNA fragments of MNase and DFF digestion products.

FIG. 2D shows the Agilent 2100 electrophoresis peak diagram of cfDNA fragments of real pregnant women's plasma.

### DETAILED DESCRIPTION OF THE INVENTION

The normal chromosome number human cell line GM12878, normal chromosome number human cell line GM23087, normal chromosome number human cell line GM23086, normal chromosome number human cell line AG09387, 21-trisomy positive human cell line AG09394, 18-trisomy positive human cell line GM02732, 13-trisomy positive human cell line GM02948, were purchased from Coriell Cell Repositories, USA.

Nonidet P-40 lysate, Sigma-Aldrich, USA

Human caspase-3 protein, Beijing Yiqiao Shenzhou Technology Co., Ltd., China

Micrococcal Nuclease (hereinafter referred to as MNase), New England Biolabs, USA

10×Micrococcal Nuclease reaction buffer, New England Biolabs, USA

Bovine serum albumin (BSA), New England Biolabs, USA

DNA fragmentation factor (DFF), recombinant DFF expression plasmid pET-15b-DFF were donated by Professor Wang Xiaodong of Beijing Institute of Life Sciences, which were expressed and purified according to the literature method listed in Example 2.

β-mercaptoethanol (2-ME), Sigma-Aldrich, USA

Ethylenediaminetetraacetic acid (EDTA), Sigma-Aldrich, USA

Glycol-bis-(2-aminoethylether)-N,N,N,N′-tetraacetic acid (EGTA), Sigma-Aldrich, USA

Mixed tablets of protease inhibitor preparation, Roche, Switzerland

Proteinase K, Life Technologies, USA

Sodium dodecyl sulfate (SDS), Sinopharm Chemical Reagent Co., Ltd., China

E. coli competent cell BL21 (pLysS), Tiangen Biochemical Technology (Beijing) Co., Ltd., China

Human plasma albumin, Sigma-Aldrich, USA

Simulated body fluid, Zhongke Maichen (Beijing) Technology Co., Ltd., China

MagMAX™ cell-free DNA extraction kit, Thermo Fisher Scientific, USA

### Example 1: Culturing Cell Lines and Extracting Nuclei

I. Method

1. Cell Culture:

The normal chromosome number human cell lines GM12878, GM23087, GM23086, and AG09387 were cultured separately, and cultured with RPIM-1640 medium containing 15% fetal bovine serum, the medium contained 15% fetal bovine serum, 100 IU/ml penicillin, and 100 IU/ml streptomycin; the suspension culture was performed in a constant temperature cell incubator with 5% CO₂ at 37° C., the cells were passaged without protease digestion, and the cells were expanded to 10′ to 10⁸ and in logarithmic growth phase. The 21-trisomy positive human cell line AG09394 was cultured with 15% fetal bovine serum RPIM-1640 medium, which contained 15% fetal bovine serum, 100 IU/ml penicillin and 100 IU/ml streptomycin; the suspension culture was performed in a constant temperature cell incubator containing 5% CO₂ at 37° C., the cells were passaged without protease digestion, and the cells were expanded to 10′ to 10⁸ and in logarithmic growth phase. The 18-trisomy positive human cell line GM02732, 13-trisomy positive human cell line GM02948, were cultured with 15% fetal bovine serum EMEM medium, which contained 15% fetal bovine serum, 100 IU/ml penicillin and 100 IU/ml streptomycin; the adherent culture was performed in a constant temperature cell incubator with 5% CO₂ at 37° C., and the cells were passaged with 0.05% trypsin digestion, and the cells were expanded to 10⁷ to 10⁸ and in logarithmic growth phase.

2. Nuclei Extraction:

For each type of the suspension cultured cells (GM12878, GM23087, GM23086, AG09387, AG09394), the cells were harvested and placed in a 15 ml culture flask in centrifuge tube, centrifuged at 1600 rpm for 6 min, sucked to remove culture solution, added with 10 ml of pre-cooled 1×PBS for washing; after thoroughly mixing, 10 μl was pipetted for counting under microscope, and about 1×10⁷ cells were pipetted and centrifuged at 300 g and 4° C. for 10 min; after removing the supernatant, the precipitated cells were resuspended in 5 ml of pre-cooled NP-40 lysate, placed on ice for 5 minutes, centrifuged at 120 g and 4° C. for 10 min, the supernatant was carefully sucked out to obtain the nuclei, which were stored at −80° C. after aliquoting.

For the adherent cultured cells (GM02732, GM02948), about 500 μl of 0.05% trypsin was pipetted and added to the culture flask for digestion until the cells become round under microscope, the cells in the culture flask were harvested and placed in a 15 ml centrifuge tube, centrifuged at 1600 rpm for 6 min, the culture solution was sucked out, 10 ml of pre-cooled 1×PBS was added for washing, after thoroughly mixing, 10 μl was pipetted for counting under microscope, and about 1×10⁷ cells were pipetted and centrifuged at 300 g and 4° C. for 10 min; after the supernatant was sucked out, the precipitated cells were resuspended in 5 ml of pre-cooled NP-40 lysate, placed on on ice for 5 min, centrifuged at 120 g and 4° C. for 10 min, the supernatant was carefully sucked out to obtain the nuclei, which were stored at −80° C. after aliquoting.

II. Results

The resultant nuclei derived from different cell lines were collected and stored at −80° C. for further preparation of cfDNA fragments by digestion with different nucleases.

### Example 2: Digestion of Nuclei by Different Enzymes to Generate Corresponding cfDNA Fragments

I. Method

1. Production of cfDNA Fragments for Stimulating Fetal Sample Cell Line by Using MNase Digestion Nuclei:

The extracted nuclei of 21-trisomy positive human cell line AG09394, 18-trisomy positive human cell line GM02732, 13-trisomy positive human cell line GM02948 and normal chromosomes number human cell line GM23086 were digested with MNase to obtain the corresponding fetal cfDNA fragments in simulated maternal plasma. Specific steps were as follows:

(1) 1 tube of extracted nuclei (about 1×10⁷ cells) was taken, and the nuclei were washed with 2 to 3 ml of MNase reaction buffer (1×Micrococcal Nuclease reaction buffer, 9% (v/v) 2-ME, 1 piece/10 ml protease inhibitor, 100 μg/ml BSA), the washing was repeated for 3 times, after centrifugation at 120 g and 4° C. for 10 min, the supernatant was carefully discarded, and the nuclei were resuspend with 100 μl of MNase reaction buffer to obtain a nuclear suspension;

(2) 300 U MNase was added to the above nuclear suspension and incubated in a 37° C. water bath for 10 min;

(3) 20 μl of MNase stop solution (250 mmol/L EDTA, 250 mmol/L EGTA) was added to the reaction tube after incubation for the corresponding time, and standing at room temperature was performed for 5 minutes;

(4) The DNA concentration in the reaction product was determined using Qubit 3.0, and the size distribution of nucleic acid fragments was observed by Agilent 2100 electrophoresis.

2. Production of cfDNA Fragments for Stimulating Maternal Cell Line by Using DFF Digestion Nuclei:

The extracted nuclei of normal chromosome number human cell line GM12878, normal chromosome number human cell line GM23087, and normal chromosome number human cell line AG09387 were digested with DFF to obtain the corresponding maternal cfDNA fragments in simulated pregnant woman's plasma.

(1) Expression and Purification of DFF Protein:

Recombinant DFF expression plasmid pET-15b-DFF (prepared according to U.S. Pat. No. 6,165,737 or the following literature method) was used to transform E. coli competent cell BL21 (pLysS). Single positive colonies were picked and cultured in LB culture medium at 37° C. with shaking overnight; 5 ml of bacterial solution was added to 300 ml of fresh LB medium and cultured at 37° C. and 220 rpm for 4 hours, and then IPTG was added to continue the shaking culture for 4 hours. The resulting culture solution was centrifuged and the supernatant was discarded. The bacterial cells were resuspended with 10% glycerol containing buffer A (20 mM Hepes-KOH, pH 7.5, 10 mM KCl, 1.5 mM MgCl₂, 1 mM sodium EDTA, 1 mM sodium EGTA, 1 mM DTT, 0.1 mM PMSF), centrifuged at 10,000 g for 30 min after ultrasonication, and the supernatant after centrifugation was purified by nickel ion column affinity chromatography; the purified DFF was obtained by elution with buffer A that contained 250 mM imidazole, and the purified DFF was verified by protein quantification and SDS-polyacrylamide gel electrophoresis (SDS-PAGE), and stored at −80° C. after aliquoting;

The expression and purification of DFF could refer to the following literature methods, which were the prior art.

(1) Widlak P, Li P, Wang X, Garrard W T. Cleavage preferences of the apoptotic endonuclease DFF40 (caspase-activated DNase or nuclease) on naked DNA and chromatin substrates. J Biol Chem 2000; 275: 8226-32. Widlak P, Li P, Wang X, Garrard W T. Study on site preference in digestion of naked DNA and chromatin substrate with apoptosis-related endogenous nuclease DNA fragmentation factor 40 (dnases or nucleases activated by apoptotic protease). Journal of Biochemistry, 2000; 275; 8226-32.

(2) Liu X, Li P, Widlak P, Zou H, Luo X, Garrard W T, Wang X. The 40-kDa subunit of DNA fragmentation factor induces DNA fragmentation and chromatin condensation during apoptosis. Proc Natl Acad Sci USA 1998; 95: 8461-6. Liu X, Li P, Widlak P, Zou H, Luo X, Garrard W T, Wang X. DNA fragmentation and chromatin condensation during apoptosis induced by 40-kilodalton subunit of DNA fragmentation factor. Journal of the National Academy of Sciences, USA, 1998; 95: 8461-6.

(2) DFF digestion of nuclei: after obtaining the purified DFF, a reaction system was prepared to digest the corresponding nuclei, and the specific steps were as follows:

\textcircled{1} 1 tube of the extracted nuclei (about 1×10⁷ cells) was taken, the nuclei were washed with 1 ml of buffer A, the washing was repeated for twice, after centrifugation at 200 g and 4° C. for 5 min, the supernatant was carefully discarded, and the nuclei were resuspended with 100 μl of buffer A;

\textcircled{2} 10 μg of dry human caspase-3 protein powder was taken, added with 105 μl of deionized water for dissolving, and a digestion reaction system (Table 1) was prepared based on the quantitative result of DFF protein concentration (0.585 mg/ml), and the reaction was performed at 37° C. for 2 hours.

TABLE 1
Reaction system for DFF digestion of cell nuclei

| Reaction component | Volume |
|---|---|
| DFF | 30 μL |
| caspase-3 | 45 μL |
| Buffer A | 28 μL |
| Nuclear solution | 10 μL |
| Total system | 113 μL |

\textcircled{3} After incubation at 37° C. for a corresponding period of time, 25 μl of reaction stop solution (0.6% SDS, 50 mM EDTA, and 6 mg/ml proteinase K) was added to the reaction tube to terminate the reaction, mixed by inversion, and incubated at 50° C. for 1 hour.

\textcircled{4} The DNA concentration in the reaction product was determined by using Qubit 3.0, and the size distribution of the nucleic acid fragments was observed by Agilent 2100 electrophoresis.

II. Results

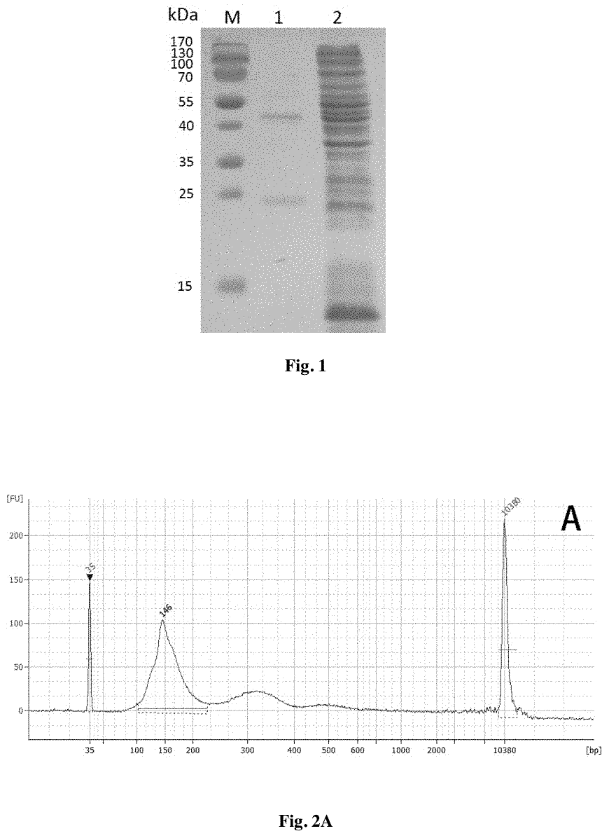

In this example, the expressed and purified DFF had a concentration of 0.585 mg/ml after protein quantification. The SDS-PAGE results were shown in FIG. 1 (wherein Lane 1 was the electrophoresis result of the purified DFF, and Lane 2 was the electrophoresis result of the unpurified DFF);

The concentrations as determined for the fetal cfDNA fragments were: AG09394 (38 ng/μl), GM02732 (28 ng/μl), GM02948 (23 ng/μl), GM23086 (26 ng/μl); the concentrations of the maternal cfDNA fragments were: AG09387 (32 ng/μl)), GM12878 (33 ng/μl), GM23087 (50 ng/μl).

### Example 3: Preparation of Reference Material for Non-Invasive Prenatal Testing

I. Method

1. Preparation of artificial plasma: a certain amount of human plasma albumin dry powder was weighed, added to a corresponding volume of the simulated body fluid at a concentration of 5%, fully dissolved and stored at 4° C.;

2. Preparation of reference material for non-invasive prenatal testing:

The chromosome aneuploid or normal chromosome number cfDNA fragments that were produced with MNase digestion and obtained in Example 2 were used as fetal cfDNA components, and separately mixed with the normal chromosome number cfDNA fragments that were produced with DFF digestion and used correspondingly as maternal cfDNA components at a certain ratio to the fetal cfDNA, then the mixed cfDNA fragments were added to the artificial plasma at a certain concentration to establish a sample tray of NIPT reference materials, and each of the sample tray contained a total of 3 chromosome aneuploid positive mixed samples and 1 normal control sample consisting of cfDNA fragments derived from a normal chromosome number human cell line;

The cell lines in the samples No. 1, 2, and 3 were not randomly matched, in which No. 1 was fetal 21-trisomy positive sample, and the two cell lines were real mother-child paired cell lines; No. 2 was fetal 18-trisomy positive sample, No. 3 was fetal 13-trisomy positive sample, and the maternal cell lines for both No. 2 and No. 3 were GM12878, while GM12878 had no relationship with these two fetal trisomy positive cell lines, and GM12878 was hereon fictitiously deemed as the maternal cell line of the 18- or 13-trisomy positive cell line for pairing.

The sample NO. 4 was a normal trisomy negative mother-child paired sample. As a negative control, these two cell lines (GM23087 and GM23086) were true mother-child paired cell lines with normal chromosome numbers. Thus, the sample No. 4 was a specific combination as a negative control.

The reasons of using a total of 4 samples were that: No. 1, 2, and 3 were trisomy 21-, 18-, and 13-trisomy positive samples, respectively, which corresponded to the three most common chromosome aneuploidies in clinical practice, and No. 4 was used as a normal negative control.

3. According to the measured concentration of each kind of cfDNA fragments, calculation was carried out so that the proportion of fetal cfDNA in each mixed cfDNA plasma sample was 8% (w/w), and the total concentration of the mixed cfDNA fragments was 25 ng/ml.

According to the concentration of each kind of cfDNA fragments as measured in Example 2, corresponding volumes of maternal cfDNA fragments and fetal cfDNA fragments were taken to form a mother-child cfDNA mixed sample, and the cfDNA mixed sample satisfied the requirement that the proportion of fetal cfDNA was 8% and the total amount of cfDNA was 25 ng, and then the prepared mother-child cfDNA mixed sample was added to 1 ml of artificial plasma to prepare a corresponding mixed cfDNA plasma sample with a concentration of 25 ng/ml.

TABLE 2
Composition of reference material sample
tray for non-invasive prenatal testing

| | Source of cell line | Chromosome | Fetal |
|---|---|---|---|
| Sample | involved in cfDNA | aneuploid | cfDNA ratio |
| No. | sample preparation | type | (%, w/w) |
| 1 | Maternal cell line: AG09387 | Chromosome 21 | 8 |
| | Progeny cell line: AG09394 | triploid | |
| 2 | Maternal cell line: GM12878 | Chromosome 18 | 8 |
| | Progeny cell line: GM02732 | triploid | |
| 3 | Maternal cell line: GM12878 | triploid | 8 |
| | Progeny cell line: GM02948 | chromosome 13 | |
| 4 | Maternal cell line: GM23087 | None (only | 8 |
| | Progeny cell line: GM23086 | containing | |
| | | normal | |
| | | chromosome | |
| | | number cfDNA) | |

4. Extraction of cfDNA from real pregnant woman's plasma and capillary electrophoresis:

Blood sample of pregnant woman was collected clinically, centrifuged at 1600 g for 10 min at 4° C., and then centrifuged at 16000 g for 10 min. After two steps of centrifugation, the supernatant was taken to obtain a plasma sample. The extraction of cfDNA was performed using the MagMAX™ cell-free DNA extraction kit, and the specific steps were as follows:

(1) 600 μl of plasma sample was taken, added in order with 12 μl of proteinase K (20 mg/ml) and 30 μl of 20% SDS, respectively, and mixed thoroughly in a water bath at 60° C. for 20 min. After the water bath was finished, the sample tube was placed on ice for 5 min to equilibrate to room temperature;

(2) Preparation of magnetic bead binding reagent mixture: 10 μl of magnetic beads were pipetted, added to 750 μl of magnetic bead binding solution and mixed thoroughly, the magnetic bead binding solution mixture was added to each sample tube, mixed by inversion, and shaken by vortexing for 10 min;

(3) The sample tube was placed on a magnetic stand for 5 min until the liquid returned to clear. The supernatant was carefully sucked out, the sample tube was tapped gently for several times, and the residual liquid at the bottom of the tube was sucked out;

(4) The magnetic beads were resuspended in 1 ml of MagMAX™ cfDNA washing solution, the washing solution containing the magnetic beads was added to a new 1.5 ml EP tube, placed on magnetic stand for 20 s, the supernatant was collected and added to the EP tube after the sample tube was re-rinsed;

(5) The EP tube containing magnetic beads was placed on magnetic stand for 2 minutes, the supernatant was carefully discarded, the EP tube was gently tapped for 5 times, and the residual liquid at the bottom of the tube was completely sucked out;

(6) 1 ml of MagMAX™ cfDNA washing solution was added again, mixed by vortexing for 30 s, the EP tube was placed on magnetic stand for 2 min, the supernatant was carefully discarded, the EP tube was gently tapped for 5 times, and the residue at the bottom of the tube liquid was thoroughly removed;

(7) 1 ml of 80% ethanol was added, mixed by votexing for 30 s, the EP tube was placed on magnetic stand for 2 min, the supernatant was carefully discarded, the EP tube was gently tapped for 5 times, and the residual liquid at the bottom of the tube was thoroughly removed;

(8) The 80% ethanol washing was repeated once;

(9) The tube cover was opened after discarding the residual liquid, and air dried for 5 min;

(10) 50 μl of MagMAX™ cfDNA eluent was added, and mixed by vortexing for 5 min;

(11) The EP tube was placed on magnetic stand for 2 minutes, and the resulting supernatant contained cfDNA;

(12) Capillary electrophoresis was performed with Aglient Bioanalyzer 2100.

II. Results

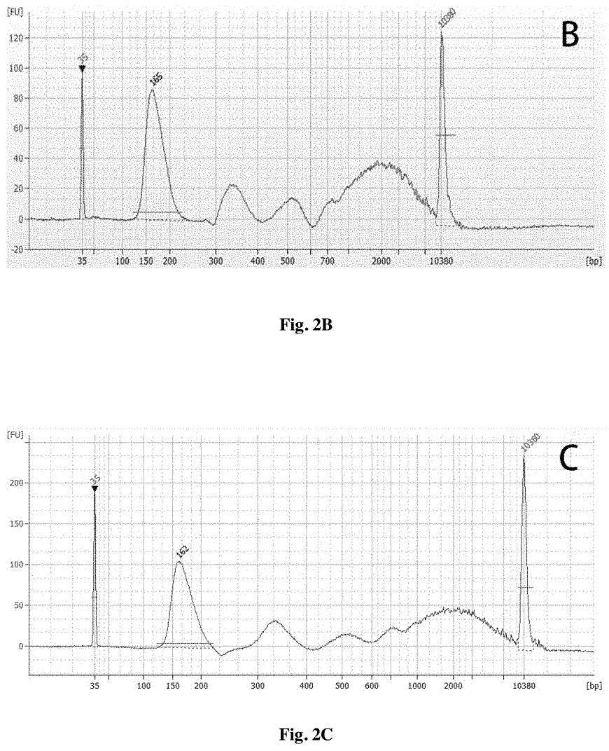

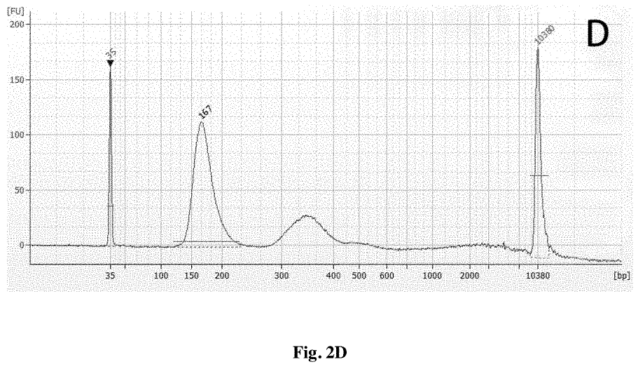

Agilent 2100 electrophoresis verification results: the capillary electrophoresis results of the cfDNA fragments produced by the digestion of the two enzymes were consistent with the expected results and similar to the distribution characteristics of real plasma cfDNA fragments. The results of cfDNA electrophoresis peaks were shown in FIGS. 2A to 2D.

### Example 4: Verification with Illumina Sequencing Platform for Reference Material for Noninvasive Prenatal Testing

I. Method:

Verification of using NextSeq 550AR sequencing platform

A sample tray of reference materials obtained in Example 3 (Table 2) was taken, treated as conventional clinical pregnant women's plasma samples, and subjected to routine fetal chromosome aneuploid noninvasive prenatal gene testing in Annuo Youda Gene Technology (Beijing) Co., Ltd. The testing results were obtained through cfDNA extraction, library preparation, sequencing on machine, and bioinformatics analysis.

The range and corresponding quality control index requirements for the conventional fetal chromosome aneuploid noninvasive prenatal gene testing in this example were shown in Table

TABLE 3

| Chromosome aneuploidy type | Fetal cfDNA ratio (w/w) | Z value |
|---|---|---|
| Chromosome 21 triploid | >3.5% | >3 |
| Chromosome 18 triploid | >3.5% | >3 |
| Chromosome 13 triploid | >3.5% | >3 |

Z Test (Z Test) is generally used to test the difference of the average of a large sample. It uses the theory of standard normal distribution to infer the probability of a difference occurring, thereby determining whether the difference between the two averages is significant by comparison. When the sample size is large and the data conform to the normal distribution, the comparison can be performed by calculating the test statistic Z-score.

In the commonly used Z-value algorithm, a sequence alignment software is used to compare the data obtained by sequencing to a human reference genome (such as NCBI build37), and the sequences of uniquely compared are used for subsequent statistics. The total valid data of large number of samples (Total mapped readsₙ) and the mapped valid data for each chromosome (Mapped to chromosomeₙₘ) were obtained, and valid data percentage (Unique reads ratio, UR %) was obtained by dividing the valid data of each chromosome by the total valid data, and the calculation formula was follows:

$${UR}_{n m} = \frac{Mapped to {chromosome}_{n m}}{Total mapped {reads}_{n }} \times 100 \%$$

wherein m was chromosome number, m∈(1 . . . 22, X, Y), and n was the number of large samples. The UR mean and variance of a large number of samples were calculated by the calculation formula as follows:

$${UR\_mean}_{m} = \frac{{\sum }_{k = 1}^{n} {UR}_{k m}}{n}$$

$${SD}_{m} = \sqrt{\frac{{\sum }_{k = 1}^{n} {( {UR}_{k m} - {UR\_mean}_{m} )}^{2}}{n}}$$

The Z value of each chromosome of each sample was calculated by the calculation formula as follows:

$${Z}_{n m} = \frac{{UR}_{n m} - {UR\_mean}_{m}}{{SD}_{m}}$$

According to statistical principles, if the Z value is a value other than plus or minus 3, there is a 99.9% chance that it will be positive, so that Z value=3 is usually set as the reference value cutoff point. Thus, fetal chromosome aneuploidy is positive when Z value >3; fetal chromosome aneuploidy is negative when Z value <3. For example, if the Z value of chromosome chr21 of a sample is greater than 3, the UR of chr21 of this sample is considered to be significant (α<0.005) outlier, that is, chr21-trisomy.

In addition, in practice, in order to achieve a certain degree of confidence, it is generally required to use a certain number of sample measurement results of true large number to set a reasonable threshold or reference value, and a gray area can also be added to help set the confidence level, and then a certain number of samples is used to verify the set Z value.

II. Results:

The test results were shown in Table 4.

TABLE 4

| | Fetal | | | | |
|---|---|---|---|---|---|
| | cfDNA | Chromosome | Z | Z | Z |
| Sample | ratio | aneuploid | value | value | value |
| no. | (%, w/w) | type | (T21) | (T18) | (T13) |
| 1 | 7.95 | Triploid of | 5.924854 | −1.088517 | −2.288213 |
| | | chromosome | | | |
| | | 21 (T21) | | | |
| 2 | 7.01 | Triploid of | −2.778662 | 5.366762 | −2.215875 |
| | | chromosome | | | |
| | | 18(T18) | | | |
| 3 | 8.08 | Triploid of | −2.334294 | −2.121936 | 4.71318 |
| | | chromosome | | | |
| | | 13 (T13) | | | |
| 4 | 9.72 | None | −1.897541 | −1.132246 | −2.513457 |

The test results of this example showed that Annuo Youda Gene Technology (Beijing) Co., Ltd. could use the NextSeq 550AR sequencing platform to detect all chromosome aneuploid types contained in the sample tray, and the reported fetal cfDNA ratios were also basically in line with the expectations, that was, the reference material used for non-invasive prenatal testing was verified by the Illumina sequencing platform. The present invention could be used as a reference material for non-invasive prenatal testing based on the Illumina sequencing platform.

### Example 5: Verification of Reference Material for Noninvasive Prenatal Testing Using Complete Genomics Sequencing Platform

I. Method:

Verification of Using BGISEQ-500 Sequencing Platform

A sample tray of reference materials obtained in Example 3 (Table 2) was taken, treated as routine clinical pregnant women's plasma samples, and subjected to routine fetal chromosome aneuploid non-invasive prenatal gene testing in Shenzhen Huada Gene Technology Co., Ltd. The testing results were obtained by cfDNA extraction, library preparation, sequencing on machine, and bioinformatics analysis.

The range and corresponding quality control index requirements for the conventional fetal chromosome aneuploid noninvasive prenatal gene testing in this example were shown in Table 5.

TABLE 5

| Chromosome aneuploidy type | Fetal cfDNA ratio (w/w) | Z value |
|---|---|---|
| Chromosome 21 triploid | >3.5% | >3 |
| Chromosome 18 triploid | >3.5% | >3 |
| Chromosome 13 triploid | >3.5% | >3 |

II. Results

The test results were shown in Table 6:

TABLE 6

| | Fetal | | | | |
|---|---|---|---|---|---|
| | cfDNA | Chromosome | Z | Z | Z |
| Sample | ratio | aneuploid | value | value | value |
| no. | (%, w/w) | type | (T21) | (T18) | (T13) |
| 1 | 9.90 | Triploid of | 6.04223 | −3.45691 | −4.34875 |
| | | chromosome | | | |
| | | 21 (T21) | | | |
| 2 | 8.45 | Triploid of | −3.12266 | 5.38672 | −3.26994 |
| | | chromosome | | | |
| | | 18 (T18) | | | |
| 3 | 9.20 | Triploid of | −3.38869 | −2.61024 | 6.63263 |
| | | chromosome | | | |
| | | 13 (T13) | | | |
| 4 | 9.94 | None | −2.8417 | −3.256 | −4.79906 |

The test results of this example showed that Shenzhen Huada Gene Technology Co., Ltd. could use the BGISEQ-500 sequencing platform to detect all chromosome aneuploid types contained in the sample tray, and the reported fetal cfDNA ratios were also basically in line with the expectations, that was, the reference material used for non-invasive prenatal testing was verified by the Complete Genomics sequencing platform. The present invention could be used as a reference material for non-invasive prenatal testing based on the Complete Genomics sequencing platform.

1. A method for preparing a simulated maternal cfDNA component for non-invasive prenatal testing, characterized in: using a DNA fragmentation factor (DFF) to digest a nucleus for simulating a maternal sample cell line, so as to obtain a simulated maternal cfDNA (short nucleic acid fragment) component.

2. The method for preparing a simulated maternal cfDNA component for non-invasive prenatal testing according to claim 1, characterized in that: the simulated maternal sample cell line is a normal chromosome number human cell line.

3. The method for preparing a simulated maternal cfDNA component for non-invasive prenatal testing according to claim 1, characterized in that: the human cell line is an immortalized cell line.

4. The method for preparing a simulated maternal cfDNA component for non-invasive prenatal testing according to claim 1, characterized in that: the method for digesting a nucleus of a maternal sample cell line comprises:
(1) taking about 1×10⁷ nuclei of the simulated maternal sample cell line, washing the nuclei with 1 ml of buffer A, repeating the washing twice, centrifuging at 200 g at 4° C. for 5 min, discarding a supernatant, and resuspending the nuclei with 100 μl of buffer A to obtain a cell nuclear solution;
the buffer A comprising components of: 20 mM Hepes-KOH (pH 7.5), 10 mM KCl, 1.5 mM MgCl₂, 1 mM sodium EDTA, 1 mM sodium EGTA, 1 mM DTT, 0.1 mM PMSF;
(2) preparing a digestion reaction system and incubating at 37° C. for 2 hours;
the digestion reaction system comprising: 30 μL of DFF solution at a concentration of 0.585 mg/ml, 45 μL of human caspase-3 at a concentration of 95 ng/μL, 28 μL of buffer A, and 10 μL of the nuclear solution obtained in step (1);
(3) adding 25 μl of a reaction stop solution to terminate the reaction, mixing by inversion, and incubating at 50° C. for 1 h; the stop solution comprising components of: 0.6% SDS, 50 mM EDTA, and 6 mg/ml proteinase K;
(4) determining a DNA concentration in the reaction product and observing the size distribution of nucleic acid fragments by capillary electrophoresis.

5. The method for preparing a simulated maternal cfDNA component for non-invasive prenatal testing according to claim 1, characterized in that: the simulated maternal cfDNA has a length of about 160 bp, which is close to the length of the maternal cfDNA in the clinical pregnant woman's plasma that is 160-170 bp.

6. A method for preparing a reference material for non-invasive prenatal testing, characterized in: digesting a nucleus of a simulated fetal sample cell line with a micrococcal nuclease (MNase) to obtain a simulated fetal cfDNA component; digesting a nucleus of a simulated maternal sample cell line with a DNA fragmentation factor (DFF) to obtain a simulated maternal cfDNA component; mixing the simulated fetal cfDNA component and the simulated maternal cfDNA component at a mass ratio of 1:99 to 30:70 to obtain a simulated mixed cfDNA component; adding the simulated mixed cfDNA component to an artificial plasma to make a reference material with a simulated mixed cfDNA concentration of 1 to 100 ng/ml.

7. The method for preparing a reference material for non-invasive prenatal testing according to claim 6, characterized in that: the simulated maternal sample cell line is a normal chromosome number human cell line, and the simulated fetal sample cell line is a chromosome aneuploid-positive human cell line or a normal chromosome number human cell line.

8. The method for preparing a reference material for non-invasive prenatal testing according to claim 6, characterized in that: the human cell line is an immortalized cell line, and the artificial plasma is a simulated body fluid containing a human plasma albumin at a concentration of 5%.

9. The method for preparing a reference material for non-invasive prenatal testing according to claim 6, characterized in that:
the method for digesting the nucleus of the simulated fetal sample cell line with micrococcus nuclease comprises the follow steps:
(1) taking 1×10⁷ nuclei of the simulated fetal sample cell line, washing the nuclei with 2 to 3 ml of MNase reaction buffer, repeatedly washing for 3 times, centrifuging at 120 g at 4° C. for 10 min, discarding the supernatant, and using 100 μl of MNase reaction buffer to resuspend the nuclei to obtain a nuclear suspension;
the MNase reaction buffer comprising components of: 1× Micrococcal Nuclease reaction buffer, 9% (v/v) 2-ME, 1 tablet/10 ml protease inhibitor, 100 μg/ml BSA;
(2) adding 300 U of MNase to the above nuclear suspension and incubating in a 37° C. water bath for 10 min;
(3) adding 20 μl of MNase stop solution to the reaction tube, standing at room temperature for 5 min; the MNase stop solution having components of 250 mmol/L EDTA, 250 mmol/L EGTA;
(4) determining the DNA concentration in the reaction product, and observing the size distribution of the nucleic acid fragments by capillary electrophoresis;
the method for digesting the nucleus of the simulated maternal sample cell line comprises the following steps:
(1) taking about 1×10⁷ nuclei of the simulated maternal sample cell line, washing the nuclei with 1 ml of buffer A, repeating the washing twice, centrifuging at 200 g at 4° C. for 5 min, discarding the supernatant, and resuspending the nuclei with 100 μl of buffer A to obtain a nuclear solution;
the buffer A comprising components of 20 mM Hepes-KOH (pH 7.5), 10 mM KCl, 1.5 mM MgCl₂, 1 mM sodium EDTA, 1 mM sodium EGTA, 1 mM DTT, 0.1 mM PMSF;
(2) preparing a digestion reaction system and incubating at 37° C. for 2 hours;
the digestion reaction system comprising: 30 μL of DFF solution at a concentration of 0.585 mg/ml, 45 μL of human caspase-3 at a concentration of 95 ng/μL, 28 μL of buffer A, and 10 μL of the nuclear solution obtained in step (1);
(3) adding 25 μl of reaction stop solution to terminate the reaction, mixing by inversion, and incubating at 50° C. for 1 h; the stop solution comprising components of 0.6% SDS, 50 mM EDTA, 6 mg/ml proteinase K;
(4) determining the DNA concentration in the reaction product and observing the size distribution of the nucleic acid fragments by capillary electrophoresis.

10. A reference material for non-invasive prenatal testing prepared by claim 6, characterized in that: the simulated maternal sample cell line is one or more selected from normal chromosome number human cell line GM12878, normal chromosome number human cell line GM23087, and normal chromosome number human cell line AG09387; the simulated fetal sample cell line is one or more selected from 21-trisomy positive human cell line AG09394, 18-trisomy positive human cell line GM02732, 13-trisomy positive human cell line GM02948, and normal chromosome number human cell line GM23086.

11. A reference material for non-invasive prenatal testing, characterized in that: which is composed of 3 positive mixed samples and 1 normal control sample; each positive mixed sample and normal control sample are composed of a simulated maternal cfDNA component, a simulated fetal cfDNA component and an artificial plasma, wherein the mass percentage ratio of the simulated fetal cfDNA component to the simulated maternal cfDNA component is 1:99 to 30:70, the artificial plasma is a simulated body fluid containing human plasma albumin at a concentration of 5%, and the concentration of the sum of the simulated fetal cfDNA component and the simulated maternal cfDNA component in artificial plasma is 1 to 100 ng/ml;
the 3 positive mixed samples are 21-trisomy positive mixed sample, 18-trisomy positive mixed sample and 13-trisomy positive mixed sample, respectively; in the 21-trisomy positive mixed sample, the simulated maternal cfDNA component is cfDNA of normal chromosome number human cell line AG09387 obtained by DFF digestion, and the simulated fetal cfDNA component is cfDNA of 21-trisomy positive human cell line AG09394 obtained by MNase digestion; in the 18-trisomy positive mixed sample, the simulated maternal cfDNA component is cfDNA of normal chromosome number human cell line GM12878 obtained by DFF digestion, and the simulated fetal cfDNA component is cfDNA of 18-trisomy positive human cell line GM02732 obtained by MNase digestion; in the 13-trisomy positive mixed sample, the simulated maternal cfDNA component is cfDNA of normal chromosome number human cell line GM12878 obtained by DFF digestion, and the simulated fetal cfDNA is cfDNA of 13-trisomy positive human cell line GM02948 obtained by MNase digestion;
in the normal control sample, the simulated maternal cfDNA component is cfDNA of normal chromosome number human cell line GM23087 obtained by DFF digestion, and the fetal cfDNA component is cfDNA of normal chromosome number human cell line GM23086 obtained by MNase digestion.
